(19) United States
(12) Patent Application Publication
(10) Pub. No.: US 2014/0018526 A1
(43) Pub. Date: Jan. 16, 2014

(54) DNA sequence in plant caragana jubata with freeze tolerance

(71) Applicants: Pardeep Kumar Bhardwaj, Palampur (IN); Ritu Kapoor, Chharera (IN); Geetika Bhagwat, Jhansi (IN); Paramvir Singh Ahuja, Director Bunglon CSIR- INBT (IN); Sanjay Kumar, E- 8 CSIR- INBT (IN)

(72) Inventors: Pardeep Kumar Bhardwaj, Palampur (IN); Ritu Kapoor, Chharera (IN); Geetika Bhagwat, Jhansi (IN); Paramvir Singh Ahuja, Director Bunglon CSIR- INBT (IN); Sanjay Kumar, E- 8 CSIR- INBT (IN)

(21) Appl. No.: 13/507,764

(22) Filed: Jul. 27, 2012

Related U.S. Application Data
(63) Continuation-in-part of application No. 12/662,536, filed on Apr. 22, 2010, now abandoned.
Application No. 12/662,536 is a continuation of application No. 11/907,419, filed on Oct. 12, 2007, now abandoned.
Application No. 11/907,419 is a continuation of application No. 11/304,613, filed on Dec. 16, 2005, now abandoned.
Application No. 11/304,613 is a continuation of application No. 10/106,799, filed on Mar. 27, 2002, now abandoned.
(60) Provisional application No. 60/279,426, filed on Mar. 29, 2001.

(51) Int. Cl. C07H 21/04

(52) U.S. Cl. CPC C07H 21/04
USPC 536/23.1

(57) ABSTRACT

An isolated DNA sequence set forth in SEQ ID NO: 33, which is differentially expressed in apical buds of plant Caragana jubata (Pall.) under freezing conditions, is disclosed.

### CROSS-REFERENCE TO RELATED APPLICATIONS

This is a continuation-in-part of Ser. No. 12/662,536, filed Apr. 22, 2010, which is a continuation of application Ser. No. 11/907,419, filed Oct. 12, 2007, which is a continuation of application Ser. No. 11/304,613, filed Dec. 16, 2005, which is a continuation of Ser. No. 10/106,799, filed Mar. 27, 2002, which claims priority on prior U.S. Provisional Application Ser. No. 60/279,426, filed Mar. 29, 2001, all incorporated herein in their entirety by reference.

### REFERENCE TO SEQUENCE LISTING

The present application incorporates by reference a file named: 673-new.ST25 including SEQ ID NO.: 1 to SEQ ID NO.: 32, provided in a computer readable form—on a diskette, created on Oct. 22, 2002 and containing 7,860 bytes. The sequence listing information recorded on the diskette is identical to the written (on paper) sequence listing provided herein.

### FIELD OF INVENTION

The present invention relates to three novel sequences of SEQ ID Nos. 30-33, differentially expressed in apical buds of plant Caragana jubata (Pall.) under freezing conditions and a method of identifying differential expression in said plant species, and also, a method of introducing said sequences into a biological system to develop freeze tolerance in them.

### BACKGROUND AND PRIOR ART REFERENCES TO THE INVENTION

Low temperature is an important environmental variable limiting (a) plant growth, development and performance: (b) crop productivity; and (c) plant distribution. According to a statistics, 64% of the Earth's mass experiences a temperature below 0° C. (Larcher. W. and Bauer. H.1981. Ecological significance of resistance to low temperatures, pp 403-437 Encyclopaedia of Plant physiology Vol 12 A).

Apart from other parts of the globe, such low temperatures are dominantly prevalent in Antarctic, Siberia, Alaska, northwestern Canada, polar regions, peak regions of high mountains and cold desert areas (for example, Ulaanbatar desert of Mongolia, which is a major part of 1,30,000 Km² of Gobi desert; Mojave desert with 65,000 Km² situated in intermountain zone of North America [Larcher. W. and Bauer. H.1981. Ecological significance of resistance to low temperatures, pp 403-437 Encyclopaedia of Plant physiology Vol 12 A and reference mentioned therein; Encyclopaedia Britannica Inc. 1987. 1023-1024]. In spite of freezing temperatures, floral population, though scanty, is present in some of these areas. This poses the question on the adopted adaptive mechanism of the plants in response to sub-zero temperatures. Simultaneously, such a situation offers opportunity to exploit the genetic make up of the plant responsible for adaptation under such harsh environmental condition.

In many species of higher plants, a period of exposure to low non-freezing temperatures results in an increased level of freezing tolerance (Thomashow, M. F. 1990. Adv. Genet. 28: 99-131). Considerable effort has been directed at to understand the molecular basis of this cold acclimation response, yet the mechanism remains poorly understood. A large number of biochemical changes have been shown to be associated with cold acclimation including alterations in lipid composition, increased sugar and soluble protein content, and the appearance of new isozymes [Thomashow. M. F. 1990. Adv. Genet. 28: 99-131; Steponkus. P. L. Cold acclimation and freezing injury from a perspective of the plasma membrane In Katterman, F. (ed), Environmental Injury to Plants pp 1-16. Academic Press. San Diego (1990)].

Among the above parameters, alterations in proteins and lipid composition was found to be critical. Data on rye suggested that specific changes in the phospholipid composition of cell plasma membranes dramatically altered the cryobehavior of the membranes and contributed directly to the increased freezing tolerance of acclimated cells (Steponkus. P. L., Uemura., M. Balsamo. R. A., Arvinte. T. A. and Lynch. D. V. 1988. Proc. Natl. Acad. Sci. USA 85: 9026-9030).

The role of cold induced proteins as cryo-protectants has been put froward. Cold acclimated spinach and cabbage, but not non-acclimated plants, synthesized hydrophilic. heat-stable, low molecular weight polypeptides (10-20 kd) that have cryo-protective properties. In particular, these polypeptides were found to be more than 10.000 times (molar basis) effective than the low molecular weight cryoprotectants such as sucrose in protecting thylakoid membranes against freezing damage in an in vitro assay (Volger. H. G. Heber, U. 1975. Biochim. Biophys. Acta 412: 335-349: Hincha, D. K., Heber. U., Schmitt. J. M. 1989. Plant Physiol. Biochem. 27: 795-801; Hincha. D. K. Heber, U., Schmitt. J. M. 1990. Planta 180: 416-419).

Since the suggestion of Weiser (Weiser. C. J. 1970. Science 169: 1269-1278) that cold acclimation might involve changes in gene expression, a number of studies indeed established the changes in gene expression during cold acclimation in a wide range of plant species (Thomashow. M. F. 1990. Adv. Genet. 28: 99-131; Thomashow. M. F., Gilmour. S. Hajela. R., Horvath. D., Lin, C. and Guo. W. 1990. In “Horticultural Biotechnology” (A. B. Bennett. ed.) Lisa. New York. pp. 305-314). Work on the model plant arabidopsis showed that upon exposure of the plant to low non-freezing temperatures (i.e. acclimatized), it becomes more tolerant to freezing temperatures. Changes in gene expression occurred during the acclimation process (Gilmour. S. J. Hajela. R. K. and Thomashow. M. F. 1988. Plant Physiol. 87: 745-750).

The polypeptides with molecular mass of 160. 47. 24. and 15 kDa were synthesized, which remained soluble upon boiling in aqueous solution (Lin. C. Guo, W. W. Everson. E. Thomashow. M. F. 1990. Plant Physiol. 94: 1078-1083). The cold regulated gene (hereinafter referred to COR) from wheat was also found to encode “boiling-stable” polypeptides and it was related to arabidopsis COR47. a cold-regulated gene that encodes a 47 kDa boiling-stable polypeptide (Lin. C. Guo. W. W., Everson. E. Thomashow. M. F. 1990. Plant Physiol. 94: 1078-1083). These boiling-stable COR polypeptides of arabidopsis and wheat were thought to have a fundamental role in plants acclimatizing to cold temperatures (Lin. C., Guo. W. W., Everson, E., Thomashow. M. F. 1990. Plant Physiol. 94: 1078-1083). It was speculated that these polypeptides might be analogous to the cryoprotective polypeptides as reported earlier (Volger, H. G., Heber. U. 1975. Biochim. Biophys. Acta 412: 335-349; Hincha. D. K. Heber, U., Schmitt. J. M. 1989. Plant Physiol. Biochem. 27: 795-801; Hincha. D. K., Heber. U., Schmitt. J. M. 1990. Planta 180: 416-419).

Strong evidences suggested regulation of at least some of the COR genes by calcium. (Monroy. A. F., Sarhan. F. Dhindsa, R. S. 1993. Plant Physiol. 102: 1227-1235; Monroy. A. F. and Dhindsa, R. S. 1995. Plant Cell. 7: 321-331). It was shown that, in alfalfa, calcium chelators and calcium channel blockers prevented low temperature induction of COR genes. Calcium ionophores and calcium channel antagonists induced expression of COR genes at normal growth temperatures.

Similarly, cold-induced expression of the arabidopsis COR gene KIN 1 is inhibited by calcium chelators and calcium channel blockers (Knight, H. Trewavas, A. J., Knight, M. R. 1996. Plant Cell 8: 489-503). These results suggested that low temperature triggered an influx of extracellular calcium that activated a signal transduction pathway to induce the expression of COR genes. Consistent with this notion was the finding that low temperature evoked transient increases in cytosolic calcium levels in plants (Knight, M. R. Campbell. A. K. Smith. S. M. Trewavas. A. J. 1991. Nature 352: 524-526: Knight, R. Trewavas. A. J. Knight. M. R. 1996. Plant Cell 8: 489-503). In addition, low temperatures was shown to stimulate the activity of mechano-sensitive calcium-selective cation channels in plants (Ding. J. P. and Pickard. B. G. 1993. Plant J. 3: 713-720).

Recent efforts led to the identification of the C-repeat-drought responsive elements abbreviated as DRE. a cis-acting cold-regulatory element (Yamaguchi-Shinozaki, K., Shinozaki. K. 1994. Plant Cell 6: 251-264: Baker. S. S. Wilhelm. K. S., Thomashow. M. F. 1994. Plant Mol. Biol. 24: 701-713; Jiang. C. Betty Lu. and Singh, J. 1996. Plant Mol. Biol. 30: 679-684). The element, which has a 5 base pair core sequence for CCGAC, is present once to multiple times in all plant cold-regulated promoters that have been described to date; these include the promoters of the COR15a (Baker. S. S Wilhelm. K. S. Thomashow. M. F. 1994. Plant. Mol. Biol. 24: 701-713), COR78/RD29A (Horvath. D. P. McLamey, B. K. Thomashow, M. F. 1993. Plant Physiol. 103: 1047-1053; Yamaguchi-Shinozaki. K., Shinozaki. K. 1994. Plant Cell 6: 251-264). COR6.6 (Wang, H. Datla. R. Georges. F. Loewen. M. Cutler. A. J. 1995. Plant Mol. Biol. 28: 605-617) and KIN1 (Wang. H. Datla. R. Georges. F. Loewen. M. Cutler. A. J. 1995. Plant Mol. Biol. 28: 605-617) genes of arabidopsis. and the BN115 gene of Brassica napus (White. T. C. Simmonds. D. Donaldson. P. Singh. J. 1994. Plant Physiol. 106: 917-928). Deletion analysis of the arabidopsis COR15a gene suggested that the CCGAC sequence, designated the C-repeat, might be part of a cis-acting cold-regulatory element (Baker. S. S. Wilhelm. K. S. Thomashow, M. F. 1994. Plant Mol. Biol. 24: 701-713).

Three cold acclimation specific (hereinafter known as CAS) gene-clones isolated from alfalfa, were shown to be specifically expressed under cold stress and were found to display a high degree of positive correlation of their expression with the freezing tolerance levels of four cultivars of alfalfa. It has been implicated that these CAS sequences might be involved in the development of freezing tolerance in alfalfa (Mohapatra. S. S. Wolfraim. L. Poole. R. J. and Dhindsa. R. S. 1989. Plant Physiol. 89: 375-380). Changes in the freezing tolerance of alfalfa plants when cold acclimated for different time periods led to changes in the transcript levels of cas 15. a cold acclimation specific cold induced gene, isolated from alfalfa, encodin a 14.5 kD protein.

Chen and Gusta (Chen. T. H. H. and Gusta L. V. 1983. Plant Physiol. 73: 71-75.) hypothesized that ABA may be substituting for low temperature induction of cold acclimation on the basis of their observation that when the micro molar quantities of ABA were added to the suspension cell cultures of wheat, rye and bromegrass. there was significant increase in the cold hardiness level of the cells.

An analysis of in-vivo labeled soluble proteins through two-dimensional gel electrophoresis in arabidopsis showed that ABA can substitute for low temperature acclimation and induce freezing tolerance by synthesizing certain proteins which were also induced by low temperature treatment (Lang, V., Heino. P. and Palva, E. T. 1989. Theo. Appl. Genet. 77: 729-734).

During a comparison between the ABA-induced and cold-acclimation induced freezing tolerance in two cultivars of alfalfa, it was concluded that ABA did provide increased freezing tolerance to some extent as was apparent from the analysis of in-vivo labeled proteins of ABA treated seedlings through the changes in their protein profiles (Mohapatra. S. S. Poole R. J., and Dhindsa. R. S. 1988. Plant Physiol. 87: 468-473).

To exploit the advantages of the cloned low temperature related gene, transgenic approach was adopted to enhance low temperature tolerance in the transgenic plant. The following table 1 shows tolerance acquired by transgenic plants upon transformation with various gene(s):

Further attempts to modulate the molecular mechanism of low temperature tolerance are as follows:
(A) Guy, C. L., Haskell, D. W., Hofig, A., and Neven, L. G. in U.S. Pat. No. 5,837,545 dated Nov. 17, 1998 described nucleotide sequences that encoded either inducible or up-regulated proteins in the leaf tissue and hypocotyl of spinach during exposure to low temperature or drought stress. Specifically described in the patent was cDNA sequences designated CAP85 and CAP 160 encoding the proteins with molecular weights of 85 and 160 kDa, respectively. Inventors also described the monoclonal antibodies that specifically recognize the disclosed proteins. Using the genes cloned by the inventors, transgenic plants were produced which showed enhanced freezing tolerance or drought resistance.
(B) Griffith. M. in another U.S. Pat. No. 5,852,172 dated Dec. 22, 1998 showed a preponderance of polypeptides with antifreeze properties. These polypeptides were found to occur extracellularly and controlled the growth of ice crystal in the xylem and intercellular plant space. These polypeptides were grouped with apparent molecular weights of about 5 to 9 kD, about 9 to 11 kD, about 11 to 15 kD, about 21 to 23 kD, about 24 to 27 kD. about 30 to 31 kD, about 31 to 33 kD, about 32 to 36 kD, about 60 and 68 kD. about 89 to 100 kD and about 161 kD. Some of these polypeptides were: (a) found to be ice nucleators for developing ice crystals in extracellular spaces of plant tissue, (b) antifreeze components, which control ice crystal growth in extracellular spaces, (c) enzymes which adapted plant cell walls to function differently during formation of ice crystals in plant intercellular spaces. Inventor proposed the development of antibodies to one or more of the polypeptides to be used as a probe for determining if a plant is frost tolerant. Inventor also proposed the use of one or more of the these polypeptides (a) to be included in frozen food preparations, particularly, in ice-cream and fruit preparations to provide a superior product having minute crystalline structure, (b) in the cryopreservation of biological tissues, (c) for long term frozen storage of a variety of tissues and frozen germplasm storage.
(C) Ekramoddoullah. A. K. M. in U.S. Pat. No. 5,686,249 described a method of determining frost hardiness of a conifer seedling by monitoring a protein of approximately 19 kD that increased significantly in amount during autumnal months and which imparted frost hardiness to the seedling N-terminal sequence of the protein in sugar pine (Finns lambertiana) which was as provided in SEQ ID NO: 1, recorded with the Protein Identification Resource Database (PIR) of the National Biomedical Research Foundation. Georgetown University Medical Centre. 3900 Reservoir Road. Washington D.C. 20007-2195. under Accession No. A 40451. since about Dec. 30, 1991]; in the case of western white pine Pinus monticola, N-terminal sequence of the cold protein was as provided in SEQ ID NO: 3. In other Finns species, a homologue (about 80% similarity) of the N-terminal sequences, mentioned as above, was detected.
(D) Thomashow. M. F. in U.S. Pat. No. 5,296,462 described the use of a polypeptide derived from a RNA encoded by a cDNA of Arabidopsis thaliana designated as COR 15 to prevent freezing or heat damage. The COR 15 is a 15 kilodalton polypeptide that is cryoprotective to chemical and biological materials.
(E) Sarhan, F., Houde. M. and Laliberte. Jean-Francois in yet another U.S. Pat. No. 5,731,419 dated Mar. 24, 1998 described the identification of a up-regulated wheat protein family which is induced by low temperature and was found it to be expressed only in freezing tolerant gramineae species. Described in the invention are three novel genes, namely Wcs 19. Wcs 120 and Wcor 410 that have been isolated from cold-tolerant wheat species. Wcs 19 requires both light and low temperature for maximal induction and is preferentially expressed in green leaf tissues of tolerant gramineae species. Wcs 120, is induced only by low temperature. Unlike the protein encoded by Wcs 19. the light-independent protein encoded by Wcs 120 consists of two repeated domains, which are highly conserved among RAB (rice abscisic acid-induced) and dehydrin families. The Wcs 120 protein does not however contain a serine-rich sequence present in RAB and dehydrin families. Wcor 410 is induced, in a light independent manner by low temperature, water stress and ABA. The protein encoded by this gene contains a serine-rich stretch, which is a general feature of several drought-induced proteins.
(F) Thomashow. M. F., Stockinger. E. J. Jaglo-Ottosen. K., Zarka. D. Gilmour. S. J. in U.S. Pat. No. 5,891,859 dated Apr. 6, 1999 described a gene. CBF1 that encodes a protein, designated as CBF 1. The protein binds the regulatory regions of genes which are activated during acclimation to low temperature and drought.
(G) Shin. C. C. Faystritsky. N. A. Sanders B. M. in U.S. Pat. No. 5,244,864 dated May 23, 1995 described the method for the protection of plant tissues from damage upon exposure to chilling temperatures and to assist plant tissues in recovering from chilling injuries by the spray application of anti chilling aqueous solutions selected from the groups consisting of tetrahydrofurfuryl alcohol, tetrahydrofurfuryl amine and mixtures thereof. The antichilling solutions appears to protect the meristem. thus leading to better growth and development during post stress periods, hence high level of survival in bean plants. In pepper plants there was significant protection of terminal buds from chilling injuries in terms of better development of terminal flower buds, quantity and quality of fruits.
(H) Caple. G. Flagstaff. A. Z., Layton. R. G. Flagstaff. A. Z. in U.S. Pat. No. 4,601,842 showed the prevention of frost injuries to the plants at moderate super cooling using aqueous solution biogenic ice nucleation inhibitor derived from various plant sources which are exposed to freezing stress in their natural environment Inhibitor inhibits the ice nucleating activity of ice nucleating bacteria, thereby reducing the temperature at which frost injury occurs.
(I) Kozloff. L. M. Schnell. R. C. in U.S. Pat. No. 4,375,734 described yet another method for the protecting plants against frost injury by using aqueous suspension of ice nucleation-inhibiting species-specific bacteriophages, whereby the frost sensitive plants are protected against frost injuries by the application of virulent bacteriophages, which selectively attack the ice nucleating bacteria, inhibiting theirice nucleation capability and hence reduce the temperature at which the frost injuries to the plants occurs. (J) Youngman, E. A. Schnell, R. C. in U.S. Pat. No. 4,311,517 dated Jan. 19, 1982 described another method of reducing the effect of freezing injuries in the cold sensitive plants eliminating the ice nucleating bacteria by treating them with one or more certain cationic quaternary ammonium surfactants. Below is specifically given a state of art knowledge with reference to cloning of low temperature related genes:

Reference may be made to document (1) by Yamaguchi-Shinozaki, K. and Shinozaki. K. 1994. Plant Cell. 6: 251-264. wherein is described the identification of a novel cis-acting element involved in responsiveness to drought, low temperature, or high salt stress from a model plant arabidopsis.

Reference may be made to document (2) by Kadyrhzhanova. D. K. Kvlachonasios. K. E. Ververidiss, P. and Dilley. D. R. 1998. wherein differential display technique was adopted to clone chilling tolerance related cDNA from tomato fruit. The clone LeHSP 17.6 was identified and hypothesized to protect the cell from metabolic dysfunction due to chilling injury.

Reference may be made to document (3) by Li, L. g., Li., S. f, Tao. Y., and Kitagawa. Y. 2000. Plant Science 154: 43-51, wherein a novel water channel protein was cloned from rice which, was shown to be involved with the chilling tolerance in Xenopus oocytes

The drawbacks in the prior art are:
(a) Efforts to induce freezing tolerance in the plants by exposing the plants to low temperature for brief periods is not possible for the plants standing in the field.
(b) Efforts to induce freezing tolerance in the plants by spraying chemical formulations will not be environmental friendly and hence would contribute to environmental pollution.
(c) There are no gene(s) till today, which have been cloned from the plants experiencing freezing temperatures under natural conditions.
(d) Earlier work to clone the genes related to freezing tolerance focussed on domesticated plant. Compared to the tamed genome of the domesticated plant, the genome of the wild plants (wild plants in the present invention refers to the undomesticated plants, where the human intervention is minimal) growing naturally in its niche environment is expected to yield unique genes. Environment at an altitude of 4200 m in western Himalaya is extremely harsh in terms of the prevailing freezing temperatures, large variations between day and the night temperature (nights are extremely cool, where the temperatures drop down to freezing temperatures in minus range) and so on. The genetic make of the plants growing in such environment is expected to yield the gene(s) whose product will confer relatively more tolerance to the plants compared to the domesticated plants.

The above drawbacks have been eliminated for the first time in a simple and reliable manner by the present invention, which is not so obvious to the person skilled in the art.

### OBJECTS OF THE PRESENT INVENTION

The main object of the present invention is to identify novel DNA molecule responsible for freeze tolerance in plant Caragana jubata (pall.) growing under snow.

Another main object of the present invention is to develop a method of identifying differential expression of genes in caragana jubata (Pall.) growing under snow and outside conditions.

Yet another object of the present invention is to identify the DNA sequence of the nucleic acid responsible for freeze tolerance in caragana jubata.

Still another object of the present invention is to identify a plant part of caragana jubata where the DNA molecules providing freeze tolerance are expressed.

Still another object of the present invention is to develop a method of incorporating the DNA molecules into a biological system to introduce freeze tolerance.

Still another object of the present invention is the cloning of the identified 3′ ends of the differentially expressed gene(s).

Yet another object of the present invention is the sequencing of the identified 3′ ends of the cloned gene.

Yet another object of the present invention is the comparison of the sequences of the cloned genes from the gene databank.

### SUMMARY OF THE INVENTION

The present invention relates to three novel sequences of SEQ ID Nos. 30-33, differentially expressed in apical buds of plant Caragana jubata (Pall.) under freezing conditions and a method of identifying differential expression in said plant species, and also, a method of introducing said sequences into a biological system to develop freeze tolerance in them.

Accordingly, the present invention relates to three novel sequences of SEQ ID Nos. 30-33 differentially expressed in apical buds of plant Caragana jubata (Pall.) under freezing conditions and a method of identifying differential expression in the plant species, and also, a method of introducing the sequences into a biological system to develop freeze tolerance in them.

In an embodiment of the present invention, DNA sequences are expressed in gene of plants growing under freezing conditions at high altitude to tolerate stress conditions.

In another embodiment of the present invention, DNA sequences are expressed at 3′ end of genes in apical buds of plant Caragana jubata (Pall.).

In yet another embodiment of the present invention, DNA sequences are differentially expressed only in the apical buds of a plant growing under snow.

A further embodiment of the present invention includes a method of identifying differentially expressed DNA sequences in apical buds of plant Caragana jubata (Pall.) growing under freezing conditions to those growing under non-freezing conditions at high altitude.

### BRIEF DESCRIPTION OF THE ACCOMPANYING DRAWING

FIG. 1 represents Total RNA isolated from the apical buds of Caragana growing in the near vicinity but away from snow (hereinafter referred to CO) and buds of Caragana growing under snow (hereinafter referred to SN). M represents RNA marker.

FIG. 2 represents spectrum of 3′ ends of the expressed and repressed genes in CO and SN apical buds of Caragana using the primer combinations as defined at the bottom of each lane. Number on the top of each lane represents lane number. Arrow indicates differential expression.

FIG. 3 represents further spectrum of 3′ ends of the expressed and repressed genes in CO and SN apical buds of Caragana using the primer combinations as defined at the bottom of each lane. Number on the top of each lane represents lane number. Arrow indicates differential expression.

FIG. 4 represents amplification of the differentially expressed 3′ ends of the gene after eluting from the denaturating polyacrylamide gel. The first number at the top of each lane represents the lane number as mentioned in FIGS. 2-3. The second number followed by the dot represents the number of differentially expressed band as counted from the top of the respective lane as mentioned in FIGS. 2-3. M represents DNA size marker.

FIG. 5 represents amplification after cloning of the eluted differentially expressed 3′ ends of the gene as mentioned in FIG. 4. The first number at the top of each lane represents the lane number as mentioned in FIGS. 2-3. The second number followed by the dot represents the number of differentially expressed band as counted from the top of the respective lane as mentioned in FIGS. 2-3. M represents DNA size marker.

FIG. 6 represents Confirmation of differential expression through northern hybridization of the cloned 3′ ends of the gene.

FIG. 7 represents expression of gene (SEQ ID NO.1) in wild type (WT) and transgenic lines (NG3, NG15) of Arabidopsis.

FIG. 8 represents effect of low temperature on fresh weight [FW] and dry weight [DW] of wild type (WT) and transgenic lines (NG3, NG15) of Arabidopsis.

FIGS. 9A-B represents effect of cold stress on root length of transgenic plants (A) 4° C. (B) 20° C.

FIGS. 10A-G represent effect of dehydration stress on seed germination.

FIGS. 11A-B represent effect of salt stress on germination.

FIGS. 12A-B are pictures showing root growth at 15° C. after (A) 1 Week, (B) 2 Weeks.

FIGS. 13A-B represent root length at 15° C. after (A) 1 Week (B) 2 Weeks.

FIGS. 14A-B are pictures showing root growth at 22° C. after (A) 1 Week, (B) 2 Weeks.

FIGS. 15A-B represent root length at 22° C. after (A) 1 Week (B) 2 Weeks.

### DETAILED DESCRIPTION OF THE INVENTION

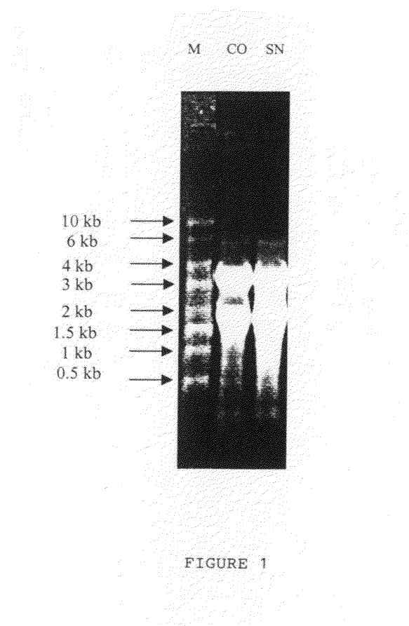

An embodiment of the present invention includes isolating total mRNA from a plant growing both under snow and outside conditions. Please refer to FIG. 1.

Another embodiment of the present invention includes reverse transcripting the mRNAs to obtain corresponding cDNA.

Yet another embodiment of the present invention includes sequencing the cDNA.

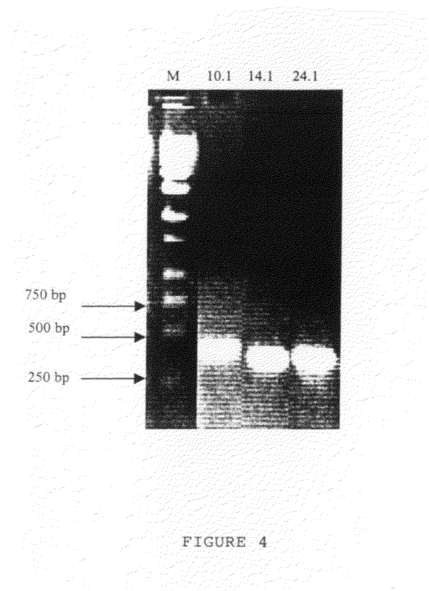

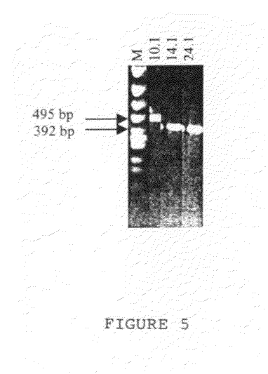

Still yet another embodiment of the present invention includes identifying differentially expressed genes using the cDNA sequences. (Please refer to FIGS. 4 and 5)

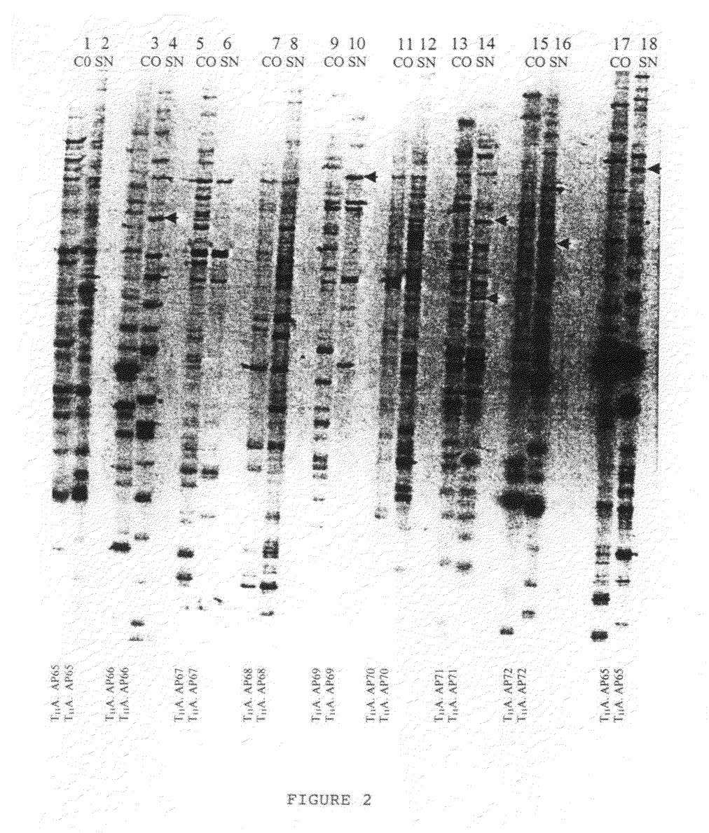

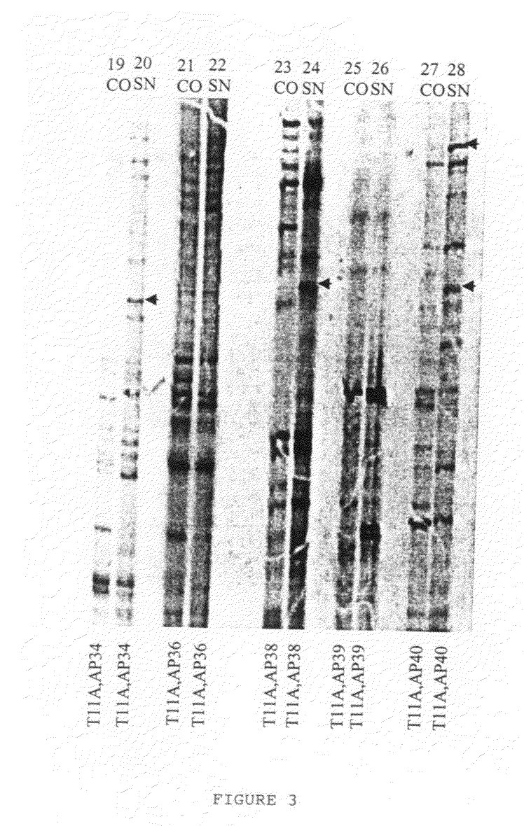

Still yet another embodiment of the present invention includes a method which shows differential expression at 3′ end of mRNA strands of the plant. (Please refer to FIGS. 2 and 3)

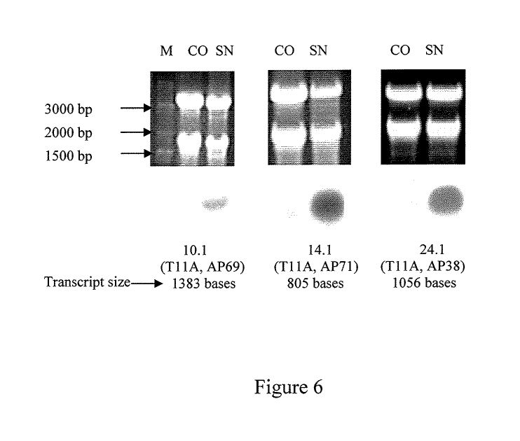

In still another embodiment of the present invention the differential expression is confirmed by Northern blotting. (Please refer to FIG. 6)

In still another embodiment of the present invention, the DNA sequences are used to develop probes to identity plants, animals, and/or microbial systems with tolerance to grow under freezing conditions.

In a further embodiment of the present invention, a method of introducing freeze tolerance in plants, animals, and/or microbial systems, includes using DNA sequences of the invention individually and in various combinations, by transferring the DNA sequences into the same.

In still another embodiment of the present invention, the method involves transferring the DNA sequences using one of the techniques known as Agrobacterium mediated transformation, and biallistic mediated transformation.

In still another embodiment of the present invention, the method is used to modulate freeze tolerance.

In further embodiment of the present invention, the present invention relates to cloning of novel genes expressed in the apical buds of Caragana jubata (Pall.) Poir (hereinafter referred to Caragana) growing under snow. Particularly, this invention relates to the comparison of gene expression pattern in the apical buds of Caragana plants growing under snow versus the Caragana plants growing in the near vicinity away from the snow with a view to identify and clone the differentially expressed gene(s). Caragana species selected in this invention were those which were growing in its niche environment at an altitude of 4200 m in western Himalaya (32° 20′ 11 “N, 78° 00' 52” E).

Particularly, this invention relates to identification, cloning and analysis of novel 3 prime (hereinafter referred to 3′) ends of the genes [gene within the present scope of invention refers to that part of deoxyribo nucleic acid (hereinafter referred to DNA) that give rise to messenger ribonucleic acid (hereinafter referred to mRNA)] expressed in apical buds of Caragana growing under snow. 3′ end refers to that end that is very close to poly A tail of mRNA.

In another embodiment of the present invention Caragana plant growing in its niche environment of western Himalaya (32° 20′ 11 “N, 78° 00' 52” E; altitude 4200 m) near a village called Kibber of Kaza town in Lahaul and Spiti district of—Himachal Pradesh was selected. When visited the area at appropriate time periods such as during the last week of March or 1^{st} week of April, it is possible to locate the plants of Caragana showing the sign of growth under the snow. The location as mentioned in the present invention experiences heavy snow-fall from the month of October onwards so as to cover the vegetation of the area. Snow starts melting from the month of March onwards and some of the plants, such as that mentioned in the present invention, start growing while still under the snow. Such a feature is exhibited by other plants such as, but not limited to, Geum species as well.

In yet another embodiment of the present invention, sign of growth is adjudged by the green-colored apical buds of the plant. Interestingly, it is possible to locate the plants in the near-by vicinity (near by vicinity in the present invention refers to a perimeter of not more than 100 meter), which also show sign of the growth, but in an open environment without snow. Thus the mentioned niche location presents the plants growing under snow (i.e. experiencing freezing temperatures) and those growing in the near by areas without snow. Such an interesting plant growing under such unique environment was exploited to identify, isolate, clone and analyze the genes expressed in the apical buds of the plants growing under the snow.

In still another embodiment apical buds were collected from the plants growing under snow and those growing in the near-by vicinity without snow. Apical buds were washed with diethyl pyrocarbonate (hereinafter known as DEPC) treated water [to prepare DEPC treated water, DEPC was added in distilled water to a final concentration of 0.1% followed by autoclaving (i.e. heating at 121° C. under a pressure of 1.1 kg per square centimeters) after an overnight incubation], harvested and immediately dipped in liquid nitrogen to freeze the cellular constituents for ceasing the cellular activities. All the collections were made on sight. In still another embodiment this invention relates to identification, cloning and analysis of novel 3 prime (hereinafter called as 3′) ends of the genes that are expressed in apical buds of Caragana growing under snow.

In still another embodiment of the present invention, total RNA from CO and SN buds was isolated and the “differential display technique” (Liang, P., Zhu. W., Zhang, X. Guo. Z. O'ConnelL R. Averboukh, L. Wang. F. and Pardee. A. B. 1994. Nucleic

Acids Res. 22: 1385-1386) was employed to generate a spectrum of 3′ ends of the expressed and repressed genes in CO and SN buds of Caragana.

In still another embodiment of the present invention, 3′ ends of the expressed genes in SN buds of Caragana were ligated into a vector to yield a recombinant plasmid. which upon transformation into a suitable E. coli host resulted into a clone. Vector, in the present invention refers to the sequence of DNA capable of accepting foreign DNA and take the form of a circular plasmid DNA that shows resistance to a given antibiotic.

Still yet another embodiment of the present invention includes novel gene sequences in the apical buds of Caragana plants growing under snow in the natural environmental conditions.

Still yet another embodiment of the present invention includes spectrum of 3′ ends of the expressed and repressed genes in the apical buds of Caragana plants growing under snow versus the Caragana plants growing in the near vicinity away from the snow under the natural environmental conditions for the purpose of identification of differentially expressed genes and cloning thereafter.

Still yet another embodiment of the present invention includes confirmation of the identified 3′ ends of the differentially expressed gene(s) for establishing differential expression in the Caragana plants growing under field conditions.

Still yet another embodiment of the present invention includes sequence information of the cloned 3′ ends of the differentially expressed gene(s).

In still another embodiment of the present invention the gene cloned was tested for its expression or repression in CO and SN buds of Caragana to define association of the cloned gene with the freezing tolerance.

In still another embodiment of the present invention the gene was sequenced using the dideoxy chain termination method (Sanger. F. S., Nicklen. and A. R. Coulson 1977. Proc. Natl. Acad. Sci. 74: 5463-5467) to figure out the uniqueness of the gene.

The present invention will be illustrated in greater details by the following examples. These examples are presented for illustrative purposes-only and should not be construed as limiting the invention, which is properly delineated in the claims.

### Example 1

### RNA Isolation, Digestion of RNA with DNase 1, Quantification of RNA and Gel-Electrophoresis

To ensure a high quality of ribonucleic acid (hereinafter known as, RNA) from CO and SN buds of Caragana. RNeasy plant mini kits (purchased from M/s. Qiagen. Germany) were used. Manufacturer's instructions were followed to isolate RNA. RNA was quantified by measuring absorbance at 260 nm and the purity was monitored by calculating the ratio of absorbance measured at 260 and 280 nm. A value >1.8 at 260/280 nm was considered ideal for the purpose of present investigation. The formula used to calculate RNA concentration and yield was as follows:

Concentration of RNA (μg/ml)=A260 (absorbance at 260 nm)×40×dilution factor

Total yield (μg)=concentration×volume of stock RNA sample

To check the integrity of RNA, 5-6 jag of RNA in 4.5 μl of DEPC treated autoclaved water was diluted with 15.5 μl of Ml solution (2 μl of 5×MOPS buffer. 3.5 μl of formaldehyde, and 10 μl of formamide [5×MOPS buffer: 300 mM sodium acetate, 10 mM MOPS (3-{N-morpholino]propanesulfonic acid}. 0.5 mM ethylene diamine tetra-acetic acid (EDTA)] and incubated for 15 minutes at 65° C. RNA was loaded onto 1.5% formaldehyde agarose-gel after adding 2 μl of formaldehyde-gel loading buffer [50% glycerol. 1 mM EDTA (pH, 8.0), 0.25% bromophenol blue. 0.25% xylene cyanol FF], and electrophoresed at 72 volts in IX MOPS buffer (60 mM sodium acetate, 2 mM MOPS. 0.1 mM EDTA), (Sambrook, J., Fritsch, E. F. and aniatis. T. 1989. Molecular Cloning: A Laboratory Manual. Cold Spring Harbor Laboratory Press, Plainview, N.Y.).

To remove the residual DNA, RNA (10-50 ug) was digested using 10 units of DNase I. in IX reaction buffer [10× reaction buffer: 100 mM Tris-Cl (pH, 8.4), 500 mM KCl, 15 mM MgCl₂, 0.01% gelatin] at 37° C. for 30 minutes (Message Clean Kit from M/s. GenHunter Corporation, USA). DNase I was precipitated by adding PCI (phenol, chloroform, isoamylalcohol in ratio of 25:24:1) and RNA present in the aqueous phase was precipitated by adding 3 volumes of ethanol in the presence of 0.3 M sodium acetate. After incubating for 3 hours at −70° C., RNA was pelleted, rinsed with chilled 70% ethanol and finally dissolved in 10 μl of RNase free water. DNA-free-RNA thus obtained was quantified and the integrity was checked as above. The quality of RNA is depicted in FIG. 1. Although we have used RNeasy columns from M/S Qiagen. Germany, the other procedure can also be used to isolate RNA from the apical buds of Caragana.

### Example 2

### Conversion of mRNA into Complementary DNAs (Hereinafter Referred to cDNAs) by Reverse Transcription (Hereinafter Referred to RT)

0.2 μg of DNA-free-RNA from CO and SN samples was reverse transcribed in separate reactions to yield cDNAs using an enzyme known as reverse transcriptase. The reaction was carried out using 0.2 μM. of T₁₁M primers (M in T₁₁M could be either T₁₁ A, T₁₁C or T₁₁G), 20 μM of dNTPs, RNA and RT buffer [25 mM Tris-Cl (pH. 8.3). 37.6 mM KCl, 1.5 mM MgCl₂ and 5 mM DTT]. In the present invention, dNTP refers to deoxy nucleoside triphosphate which comprises of deoxyadenosine triphosphate (hereinafter referred to dATP), deoxyguanosine triphosphate (hereinafter referred to dGTP), deoxycytidine triphosphate (hereinafter referred to dCTP) and deoxythymidine triphosphate (hereinafter reffered to dTTP). Three RT reactions were set per RNA sample for the corresponding T₁₁M primer. The reactions were carried out in a thermocycler (model 480 from M/s Perkin-Elmer, USA). Thermocycler parameters chosen for reverse transcription were 65° C. for 5 minutes.->37° C. for 60 minutes.->75° C. for 5 minutes.->4° C. 100 units of reverse transcriptase was added to each reaction after 10 minute incubation at 37° C. and reaction then continued for rest of the 50 minutes. Two different RNA (CO and SN) in combination with 3 T₁₁M primers yielded a total of 6 reactions depicting 6 different classes of cDNAs. The use of 3 different T₁₁M primers divided the whole RNA population into 3 sub-classes depending upon the anchored base M. which was either A, C or G (Reverse transcription system was a component of RNAimage kit from M/s. GenHunter Corporation, USA).

### Example 3

### Generation of a Spectrum of Differentially Expressed Genes Through Differential Display Technique for Identification of Differentially Expressed Gene(s)

Different sub-classes of cDNA from CO and SN RT product as obtained in Example 2 were amplified in the presence of a radiolabelled dATP to label the amplified product through polymerase chain reaction (hereinafter known as PCR; PCR process is covered by patents owned by Hoffman-La Roche Inc.). Radioactive PCR was carried out in 20 μl reaction mix containing a (1) reaction buffer [10 mM Tris-Cl (pH. 8.4). 50 mM KCl. 1.5 mM MgCl₂. 0.001% gelatin], (2) 2 μM dNTPs. (3) 0.2 μM T₁₁M and (4) 0.2 μM arbitrary primers (chemicals 1 to 4 were purchased from M/s. GenHunter Corporation, Nashville. USA as a part of RNAimage kit). 0.2 μl α[³³P] dATP (˜2000 Ci/mmole. purchased from JONAKI Center, CCMB campus Hyderabad. India), and 1.0 units of Thermus aqueticus (hereinafter referred to Taq) DNA Polymerase (purchased from M/S. Qiagen. Germany). 30 μl of autoclaved mineral oil was overlaid at the top of each reaction to avoid alteration in volume due to evaporation. T₁₁M primer in each reaction was the same that was used to synthesize cDNA. Parameters chosen were: 40 cycles of 94° C. for 30 seconds,->40° C. for 2 minutes.->72° C. for 30 seconds; and 1 cycle of 72° C. for 5 minutes and final incubation at 4° C.

Amplified products were fractionated onto a 6% denaturating polyacrlamide gel. For the purpose 3.5-μl of each of amplified product was mixed with 2 μl of loading dye [95% formamide. 10 mM EDTA (pH. 8.0). 0.09% xylene cyanol FF and 0.09% bromophenol blue], incubated at 80° C. for 2 minutes and loaded onto a 6% denaturating polyacrlamide gel [denaturating polyacrlamide gel: 15 ml of acrylamide (40% stock of acrylamide and bisacrylamide in the ratio of 20:1). 10 ml of 1 OX TBE, 40 ml of distilled water and 50 g urea]. Electrophoresis was performed using 1×TBE buffer [10×TBE: 108 g Tris base, 55 g boric acid and 40 ml of 0.5 M EDTA (pH, 8.0)] as a running buffer at 60 watts until the xylene cyanol (the slower moving dye) reached the lower end of the glass plates. Size of the larger plate of the sequencing gel apparatus was 13×16 inch. After the electrophoresis, one of the glass plates was removed and the gel was transferred onto a 3 MM Whattman filter paper. Gel was dried at 80° C. under vacuum overnight and exposed to Kodak X-ray film for 2-3 days. Before exposing to X-ray film, corners of the dried gel were marked with radioactive ink for further alignment. FIGS. 2-3 show the spectrum of differentially expressed genes in CO and SN apical buds of Caragana as was seen after developing the film. After developing the gel. film was analyzed for differentially expressed bands between CO and SN signals.

Sequences of the primers used for differential display were as follows (purchased from M/s. GenHunter Corporation, USA as a part of RNAimage kit):

| T₁₁M (anchored) primers | Primer sequence | |
|---|---|---|
| T₁₁A | 5′-AAGCTTTTTTTTTTTTTA-3′ | (SEQ ID NO: 1) |
| T₁₁C | 5′AAGCTTTTTTTTTTTTTC-3″ | (SEQ ID NO: 2) |
| T₁₁G | 5′-AAGCTTTTTTTTTTTTTG-3′ | (SEQ ID NO: 3) |
| Arbitrary Primers | Primer Sequence | |
| AP1 | 5′-AAGCTTGATTGCC-3′ | (SEQ ID NO: 4) |
| AP2 | 5′-AAGCTTCGACTGT-3′ | (SEQ ID NO: 5) |
| AP3 | 5′-A AGCTTTGGTC AG-3′ | (SEQ ID NO: 6) |
| AP4 | 5′-AAGCTTCTCAACG-3′ | (SEQ ID NO: 7) |
| APS | 5′-AAGCTTAGTAGGC-3′ | (SEQ ID NO: 8) |
| AP6 | 5′-AAGCTTGCACCAT-3′ | (SEQ ID NO: 9) |
| AP7 | 5′-AAGCTTAACGAGG-3′ | (SEQ ID NO: 10) |
| AP8 | 5′-AGCTTTTACCGC-3′ | (SEQ ID NO: 11) |
| AP33 | 5′-AAGCTTGCTGCTC-3′ | (SEQ ID NO: 12) |
| AP34 | 5′-AAGCTTCAGCAGC-3′ | (SEQ ID NO: 13) |
| AP35 | 5′-AAGCTTCAGGGCA-3′ | (SEQ ID NO: 14) |
| AP36 | 5′-AAGCTTCGACGCT-3 ′ | (SEQ ID NO: 15) |
| AP37 | 5′-AAGCTTGGGCCTA-3′ | (SEQ ID NO: 16) |
| AP38 | 5′-AAGCTTCCAGTGC-3′ | (SEQ ID NO: 17) |
| AP39 | 5′-AAGCTTTCCCAGC-3′ | (SEQ ID NO: 18) |
| AP40 | 5′-AAGCTTGTCAGCC-3′ | (SEQ ID NO: 19) |
| AP65 | 5′-AAGCTT CAAGACC-3′ | (SEQ ID NO: 20) |
| AP66 | 5′-AAGCTT GCCTTTA-3′ | (SEQ ID NO: 21) |
| AP67 | 5′-AAGCTT TATTTAT-3′ | (SEQ ID NO: 22) |
| AP68 | 5′-AAGCTT CTTTGGT-3′ | (SEQ ID NO: 23) |
| AP69 | 5′-AAGCTT AATAACG-3′ | (SEQ ID NO: 24) |
| AP70 | 5′-AAGCTT TCATATG-3′ | (SEQ ID NO: 25) |
| AP71 | 5′-AAGCTT GTAGTAA-3′ | (SEQ ID NO: 26) |
| AP72 | 5′-AAGCTTTCAAAGA-3′ | (SEQ ID NO: 27) |

Although, we used a large number of primers as shown in the above list. However, in the present document only those gels and the primer combinations, which showed confirmatory results through northern hybridization, have been shown in FIGS. 2 and 3.

### Example 4

### Re-Amplification of cDNA Probes

Cloning the differentially expressed bands required elution of the same from the denaturating polyacrylamide gel and further amplification to yield substantial quantity of DNA for the purpose of cloning. Autoradiogram (developed X-ray film) was oriented with the dried gel aided with radioactive ink. The identified differentially expressed band (along with the gel and the filter paper) was cut with the help of a sterile sharp razor. DNA was eluted from the gel and the filter paper by incubating them in 100 μl of sterile dH₂O for 10 min in an eppendorf tube, followed by boiling for 10 minutes. Paper and gel debris were pelleted by spinning at 10.000 rpm for 2 min and the supernatant containing DNA was transferred into a new tube. DNA was precipitated with 10 μl of 3M sodium acetate, pH, 5.5, 5 μl of glycogen (concentration of stock: 10 mg/ml) and 450 μl of ethanol. After an overnight incubation at −70° C., centrifugation was performed at 10,000 rpm for 10 min at 4° C. and pelleted DNA was rinsed with 85% ethanol. DNA pellet was dissolved in 10 μl of sterile distilled water.

Eluted DNA was amplified using the same set of T₁₁M and arbitrary primer that was used for the purpose of performing differential display as in the Example 3. Also, the PCR conditions were the same except that dNTP concentration was 20 μM instead of 2 μM and no isotopes was added. Reaction was up-scaled to 40 μl and after completion of PCR, 30 μl of PCR sample was run on 1.5% agarose gel in TAE buffer (TAE buffer: 0.04 M Tris-acetate, 0.002 M EDTA, pH 8.5) containing ethidium bromide (final concentration of 0.5 μg/ml). Rest of the amplified product was stored at −20° C. for cloning purposes (see FIG. 4).

### Example 5

### Cloning of Re-Amplified PCR Products

Re-amplified PCR products as obtained in example 4 were ligated in 300 ng of insert-ready vector called as PCR-TRAP® vector using 200 units of T₄ DNA-ligase in 1× ligation buffer (10× ligase buffer: 500 mM Tris-Cl. pH 7.8, 100 mM MgCl₂. 100 mM DTT. 10 mM ATP, 500 ug/ml BSA). Vector and the other chemicals required were purchased from M/s. GenHunter Corporation, Nashville, USA as PCR-TRAPS cloning system. Ligation was performed at 16° C. for 16 hours in a thermocycler model 480 from M/s. Perkin Elmer. USA. Ligation of the PCR product into a vector such as above yields to a circularized plasmid. The process of ligation of the foreign DNA. such as the PCR product in the present invention, into a suitable vector, such as PCR-TRAPS vector in the present invention, is known as cloning. There is a range of other vectors that are commercially available or otherwise that suits the cloning work of PCR products and hence may be used. The plasmid. as per the definition, is a closed circular DNA molecules that exists in a suitable host cell such as in Escsherichia coli (hereinafter referred to E. coli) independent of chromosomal DNA and may confer resistance against an antibiotic. PCR-TRAP® vector resulting plasmid confers resistance against tetracycline.

Ligated product or the plasmid needs to be placed in a suitable E. coli host for its multiplication and propagation through a process called transformation. Ligated product (10 (μl) as obtained above was used to transform 100 μl of competent E. coli cells (purchased from M/s. GenHunter Corporation USA as a part of PCR-TRAP® cloning system). Competent means the E. coli cells capable of accepting a plasmid DNA. For the purpose, ligated product and competent cell were mixed, kept on ice for 45 minutes, heat shocked for 2 minutes and cultured in 0.4 ml of LB medium (LB medium: 10 g tryptone, 5 g yeast extract. 10 g sodium chloride in 1 litre of final volume in distilled/deionized water) for 4 hours. 200 μl of transformed cells were plated onto LB-tetracyclin (for 1 litre: 10 g tryptone. 5 g yeast extract. 10 g sodium chloride, and tetracyclin added to a final concentration of 20 μ/ml) plates and grown overnight at 37° C. Colonies were marked and single isolated colony was restreaked on to LB-tetracyclin plates to get colonies of the same kind. Conferral of tetracyclin resistance to E. coli cells apparently suggests that the PCR product i.e. the identified gene has been cloned.

In whole of the above process, the selection of T₁₁M primer will amplify the poly A tail region of mRNA. Poly A tail is always attached to 3′ end of the gene and hence TnM primer in combination with an arbitrary primer would always yield 3′ region of the gene.

### Example 6

### Checking the Size of the PCR Product

Once the gene has been cloned and the E. coli has been transformed, it becomes imperative to check if the plasmid has received right size of the PCR product. This can be accomplished by performing colony PCR wherein the colony is lysed and the lysate. containing template, is subsequently used to perform PCR using the appropriate primers. Amplified product is then analysed on an agarose gel.

Colonies were picked up from re-streaked plates (Example 5) and lysed in 50 μl colony lysis buffer [colony lysis buffer: TE (Tris-Cl 10 mM, 1 mM EDTA. pH 8.0) with 0.1% tween 20] by boiling for 10 minutes. Cell debris were pelleted and the supernatant or the colony lysate containing the template DNA was used for PCR. PCR components were essentially the same as in example 4 except that in place of T₁₁M and arbitrary primers. Lgh (5′-CGACAACACCGATAATC-3′) (SEQ ID NO: 28) and Rgh (5′-GACGCGAACGAAGCAAC-3′) (SEQ ID NO: 29) primers (specific to the vector sequences flanking the cloning site) were used and 2 μl of the colony lysate was used in place of eluted DNA. Also, the reaction volume was reduced to 20 PCR conditions used for colony PCR were. 94° C. for 30 seconds.->52° C. for 40 seconds.->72° C. for 1 minute for 30 cycles followed by 1 cycle of 5 min extension at 72° C. and final soaking into 4° C. Amplified product are run on 1.5% agarose gel along with molecular weight marker and analyzed for correct size of insert. While using Lgh and Rgh flanking primers, the size of the cloned PCR product was larger by 120 bp due to the flanking vector sequence being amplified (See FIG. 5).

### Example 7

### Confirmation of the Differential Expression by Northern Blotting

PCR products cloned above represent 3′ end of the differentially expressed genes. Within the scope of the present invention, these cloned fragments of DNA will be called as genes. Since differential display invariably leads to false positives i.e. apparently differentially expressed genes (Wan, J. S, and Erlander. M. G. 1997. Cloning differentially expressed genes by using differential display and subtractive hybridization. In Methods in Molecular Biology. Vol. 85: Differential display methods and protocols. Eds. Liang, P. and Pardee. A. B. Humana press Inc. Totowa. N.J. pp. 45-68). a confirmatory test through northern analysis is mandatory to ascertain differential expression between CO and SN apical buds of Caragana. Northern analysis requires preparation of a radio-labelled probe followed by its hybridization with denatured RNA blotted onto a membrane.

Amplified products as in Example 6 were used as a probe in northern analysis. After visualising the amplified products on 1.5 agarose gel these were cut from the gel and the DNA was eluted from the gel using QIAEX II gel extraction kit from M/s. Qiagen. Germany following the manufacturer's instructions.

Purified fragments were radiolabelleled with α[³²P]dATP (4000 Ci/mmole) using HotPrime Kit from M/s. GenHunter Corporation, Nashville. USA following their instructions. Radio-labelled probe was purified using QIAquick nucleotide Removal Kit (QIAGEN, Germany) to remove unincorporated radionucleotide.

For blotting. 20 μg of RNA was run on 1.0% formaldehyde agarose gel essentially as described in Example 1. Once the run was completed, gel was washed twice with DEPC treated autoclaved water for 20 minutes each with shaking. Gel was then washed twice with 10×SSPE (10×SSPE: 1.5 M sodium chloride, 115 mM NaH₂PO₄. 10 mM EDTA) for 20 minutes each with shaking. In the mean time nylone membrane (Boehringer mannheim cat. no. #1209272) was wetted in DEPC water and then soaked in 10×SSPE for 5 minutes with gentle shaking. RNA from the gel was then vacuum-blotted (using pressure of 40 mbar) onto nylon membrane using DEPC-treated 10×SSPE as a transfer medium. Transfer was carried out for 4 hours.

Pressure was Increased to 70 mbar for 15 minutes before letting out the gel from the vacuum blotter. After the transfer, gel was removed, and the location of RNA marker was marked on the nylon surface under a UV light source. Membrane was dried and baked at 80° C. for 45 minutes. After a brief rinse in 5×SSPE (20×SSPE: 3M sodium chloride, 230 mM sodium phosphate, 20 mM EDTA) membrane was dipped into prehybridization solution (50 formamide. 0.75 M NaCl, 50 mM sodium phosphate. pH 7.4, 5 mM EDTA. 0.1% Ficoll-400, 0.1% BSA, 0.1% polyvinypyrollidone, 0.1% SDS solution and 150 ug/ml freshly boiled salmon sperm DNA) for 5 hours.

Radiolabelled probe synthesized earlier was denatured by boiling for 10 minutes followed by addition to the prehybridization solution dipping the blotted membrane. Hybridization was carried out for 16 hours. Solution was removed and the membrane was washed twice with 1×SSC (20×SSC; 3M sodium chloride and 0.3M sodium citrate dihydrate. pH. 7.0) containing 0.1% SDS at room temperature for 15 minutes each. Final washing was done at 50° C. using pre-warmed 0.25×SSC containing 0.1% SDS for 15 minutes. Membrane was removed, wrapped in saran wrap and exposed to X-ray film for 12-240 hours depending upon the intensity of the signal.

While performing northern hybridization, RNA from CO and SN apical buds are blotted on the membrane and tested for the probe of choice. FIG. 6 show the results with 3 such probes and confirm differential expression between CO and SN apical buds. Results obtained after northern hybridization using the cloned differentially expressed 3′ ends of the gene. Numbers represents cloned fragment as explained in FIGS. 4 and 5. Primer combinations used to obtain the clones are mentioned within the parenthesis.

Three genes showed confirmed differential expressions and are designated as

10.1 (T11A. AP69)—SEQ ID NO. 30

14.1 (T11A. AP71)—SEQ ID NO. 31

24.1 (T11A,AP38)—SEQ ID NO. 32

The items mentioned inside the bracket depict primers combination. The detail of these primers is mentioned in example 3. Meaning of the numbers mentioned outside the bracket is as follows: first two numbers represent the lane number as mentioned in FIGS. 2-3. The second number followed by the dot represents the number of differentially expressed band as counted from the top of the respective lane as mentioned in FIGS. 2-3.

10.1 (T11A, AP69), which is basically a 3′ end region of the gene, hybridized to the transcript of 1383 base size on northern blot as in FIG. 6.

14.1 (T11A, AP71), which is basically a 3′ end region of the gene, hybridized to the transcript of 805 base size on northern blot as in FIG. 6.

24.1 (T11A, AP 38), which is basically a 3′ end region of the gene, hybridized to the transcript of 1056 base size on northern blot as in FIG. 6.

Size of the above transcript has been measured with the help of RNA markers (Cat# R7020) purchased from M/S. Sigma chemical company, USA

### Example 8

Each clone was sequenced manually using a T7 sequenase version 2 sequencing kit from M/s. Amersham Pharmacia Biotech, USA. Sequencing primers used were [Lgh (5′-CGACAACACCGATAATC-3′) (SEQ ID NO: 28) or Rgh (5′-GACGCGAACGAAGCAAC-3′) (SEQ ID NO: 29).
(1) INFORMATION FOR SEQ ID NO: 30
   (i) SEQUENCE CHARACTERISTICS:
      (A) LENGTH: 211 base pairs
      (B) TYPE: nucleic acid
      (C) STRANDEDNESS: double
      (D) TOPOLOGY: circular
   (ii) MOLECULE TYPE: cDNA
   (iii) SEQUENCE DESCRIPTION: SEQ ID NO: 30
2) INFORMATION FOR SEQ ID NO: 31
   (i) SEQUENCE CHARACTERISTICS:
      (A) LENGTH: 180 base pairs
      (B) TYPE: nucleic acid
      (C) STRANDEDNESS: double
      (D) TOPOLOGY: circular
   (ii) MOLECULE TYPE: cDNA
   (iii) SEQUENCE DESCRIPTION: SEQ ID NO: 31
(3) INFORMATION FOR SEQ ID NO: 32
   (i) SEQUENCE CHARACTERISTICS:
      (A) LENGTH: 273 base pairs
      (B) TYPE: nucleic acid
      (C) STRANDEDNESS: double
      (D) TOPOLOGY: circular
   (ii) MOLECULE TYPE: cDNA
   (iii) SEQUENCE DESCRIPTION: SEQ ID NO: 32

### Example 9

All the sequences were searched for uniqueness in the gene databases available at URL www.ncbi.nlm.nih.gov. Using BLAST (BLAST stands for Basic Local Alignment Search Tool). The results of the search are presented in Annexure 1, Annexure 2 and Annexure 3 for Sequence ID NO: 30, Sequence ID NO: 31, and Sequence ID No: 32. It may be appreciated from the results that the sequence were found to be unique as they did not homogy >50% with any of the sequences submitted in the databases available to the public.

### Cloning of Full Length (SEQ ID NO: 33) Gene Using RACE

Rapid amplification of cDNA ends (RACE, SMART™ RACE cDNA Amplification Kit; BD Biosciences, Clontech, USA) was used to isolate full length SEQ ID NO.1 gene from Caragana jubata. RACE amplifies DNA sequences from a messenger RNA template between a defined internal site and unknown sequences of either the 3′ or 5′ end. A set of gene specific primers were used to generate 5′ and 3′ ends of the gene separately. The partial cDNA sequence (SEQ ID No; 1) was used to design two sets of primers. Primers were designed such that the amplified 5′ and 3′ ends overlap each other over a small stretch of nucleotides. For 5′ RACE, a gene specific primer (GSP1), 5′-GCCAAATAGCAACCACGTGAATCAACC 3′ for primary PCR and one nested gene specific primer (NES1), 5′-GCAACCACGTGAATCAACCCAATTGAA-3′ for secondary PCR were designed. For 3′ RACE a gene specific primer (GSP2), 5′-TTCAATTGGGTTGATTCACGTGGTTGC-3′ for primary PCR and one nested primer (NES2), 5′-TGGGTTGATTCACGTGGTTGCTATTTGG-3′ were designed. Primers were designed such that the amplified 5′ and 3′ ends overlap each other over a small stretch of nucleotides.

The cDNA for 5′-RACE was synthesized using a modified lock-docking oligo(dT) primer and SMART II A oligo (dT) primer. The modified oligo (dT) primer, termed the 5′-RACE CDS Primer (5′-CDS) has two degenerate nucleotide positions at the 3′ end.

1 μg of total RNA was reverse transcribed in separate reactions to yield 5′ and 3′ RACE ready cDNA using an enzyme known as reverse transcriptase. For 5′ cDNA synthesis, the reaction was carried out using 1 μM of 5′-CDS primer in a reaction mixture containing RNA and 1 μM SMART II oligo (dT) primer. The 3′-RACE cDNA is synthesized using a traditional reverse transcription procedure, but with a special oligo (dT) primer. This 3′-RACE CDS Primer A (3′-CDS) primer includes the lock-docking nucleotide positions as in the 5′-CDS and also has a portion of the smart sequence at its 5′ end. Sterile H₂O was added to a final volume of 5 μl for each reaction, mixed and centrifuged. The reaction mix was incubated at 70° C. for 2 mM and cooled on ice for 2 min. First-strand buffer (50 mM Tris-Cl (pH, 8.3), 75 mM KCl and 6 mM MgCl₂), 1 mM dNTPs, 2 mM DTT and reverse transcriptase were added to each reaction and incubated at 42° C. for 1.5 hr in an air incubator. Diluted the first-strand reaction product with 100 μl of Tricine-EDTA buffer (10 mM Tricine-KOH (pH 8.5), 1.0 mM EDTA) and heated tubes at 72° C. for 7 min. (Reverse transcription system was a component of SMART RACE cDNA amplification kit from BD Biosciences, USA).

Sequences of primers used for RACE were as follows (purchased from SMART ™
RACE cDNA Amplification Kit; Clontech, USA as a part of RACE Kit).

| Primer | Primer Sequence |
|---|---|
| SMART II A Oligonucleotide | 5′-AAGCAGTGGTATCAACGCAGAGTACGCGGG-3′ |
| 3′-RACE CDS Primer A (3′-CDS) | 5′-AAGCAGTGGTATCAACGCAGAGTAC(T)₃₀ N₋₁N-3′ |
| 5′-RACE CDS Primer (5′-CDS) | 5′-(T)₂₅N₋₁N-3′ |

10XUniverselLong:PrimerMixA(UPM)
5′TAATACGACTCACTATAGGGCAAGCAGTGGTATCAACGCAGAGT-3′
Short: 5′-CTAATACGACTCACTATAGGGC-3′
Nested Universal Primer A(NUP) 5′-AAGCAGTGGTATCAACGCAGAGT-3′

5′ and 3′ RACE cDNA were amplified using 0.2 μM GSP1, GSP2 primer and 1× universal primer (UPM), 0.2 mM dNTP and 1×BD polymerase mix. Thermocycler program consisted of 30 cycles of 94° C. for 30 sec, 68° C. for 30 sec and 72° C. for 3 min. The reaction was up-scaled to 50 μl and after the completion of PCR, 45 μl of PCR sample was run on 1.2% agarose gel in TAE buffer containing ethidium bromide (final concentration of 0.5 μg/ml) Rest of the amplified product was stored at −20° C. for secondary PCR if needed. Amplicons were cut from the gel and DNA was eluted from the gel using QIAEX II gel extraction kit from M/S Qiagen, Germany following the manufacturer's instructions. The purified DNA was cloned in pGEM-T easy vector (Promega, USA), plasmids were isolated using the Qiagen plasmid mini-isolation kit, and sequencing was performed using the BigDye terminator (version 3.1) cycle sequencing mix (Applied Biosystems, USA) on an automated DNA sequencer (ABI Prism 310, Genetic Analyzer, Applied Biosystems). Protocols were followed essentially as described by respective manufacturers.

Full length sequence obtained was: (1682 bp)
ACGCGGGGGAAGGAATATAGTAGCACGCAGAGGCTCTCTCTTGACTGTTG
GTTCGAATCGAAATCGAAATCAAATCCATTTTTCAATTGGATCGTCTCAG
TCTCCGATCTTCAAATCTCTAACACAATGGTGAGAGGACTGCTCAACAAG
CTCGTCTCCCGTTCCCTCTCCGTCGCTGGCAAATGGCAGAATCAACAGCT
CCGCCGCCTCAACATCCACGAGTACCAGGGAGCAGAATTGATGAGCAAAT
ACGGTGTCAACGTTCCCAGAGGTGTCGCTGTTTCCTCAATTGAAGAAGTC
AGAAAGGCCATCAAGGACGTATTCGCCAATCAAAATGAGGTGGTGGTTAA
GAGCCGAATTTTAGCTGGTGGACGAGGCTTGGGAACTTTTAAAAGTGGCC
TTCAGGGAGGAGTACACATTGTTAAGACTGACCAGGTTGAAGATTTAGCT
GGGAAAATGCTTGGGCAGATACTGGTTACCAAACAGACTGGCCCTCAGGG
GAAAGTTGTCAGCAAGGTTTACTTGTGTGAAAAGCTGTCACTTGTGAATG
AAATGTACTTTGCTATAACTCTGGATCGGAAGACTGCTGGTCCACTTATA
ATTGCTTGTAGAAAGGGAGGAACCAGCATCGAAGACCTTGCAGAGAAATT
TCCAGACATGATTATAAAGGTACCTATTGATGTTTTTGAAGGAATTACCG
ACGAAGATGCTGCAAAGGTTGTTGATGGTTTGGCTCCCAAAGGGGCTGAT
AGAAATAAATCAATTGAGCAAGTGAAGAATTTGTATAAACTCTTTGTTGA
AAGTGACTGCACTCTTTTAGAAATCAATCCCATAGCGGAGACTGCTGATA
ACCAATTGGTGGCTGCTGATGCTAAGTTGAATTTTGATGATAATGCTGCA
TATCGCCAGAAAGGGATATTCAAACTTCGTGATACAACACAAGAGGATCC
TCGAGAGGTTACTGCTGCAAAGGCAGATTTGAATTACATTGGTCTAGATG
GAGAAATTGGCTGCATGGTGAATGGAGCTGGATTAGCAATGGCTACAATG
GATATAATTAAGTTGCATGGGGGAACTCCTGCGAATTTTCTGGATGTTGG
TGGGAATGCCTCTGAGGGCCAGGTGGTTGAGGCATTTAAGATATTGACCG
CAGACGACAAAGTGGAAGCTATTTTGGTTAACATTTTTGGTGGTATAATG
AAGTGTGATGTTATAGCCAGTGGAATAGTCAATGCTGCAAAACAGGTTGC
ACTAAAAGTACCAGTGGTAGTTCGTCTTGAAGGCACCAATGTTGATCAAG
GAAAAAGAATTTTGAAGGAAAGTGGTTTGGCATTAATAACGGCTGAAGAT
TTGGATGATGCAGCTCAGAAAGCAGTGAAAGCTTACAAATGAAGGGTCAC
TTCCGTTCAAATTTAAAAATCAGTTTAGTCACAACATTATTTTTTGTGTT
AACAATTATTATTCAGAAGAGAATTGATGCTAGAAATCTTCATATTGAAG
CCGCAGTGGCTATCAAGGCGTTTCCAATAAGTTAACGCTTTTTTCAAATT
TTAGTTCAATTGGGTTGATTCACGTGGTTGCTATTTGGCTTCTCTCTTGC
AACTCAAGATACTGTCCCTTTCCTGGTTAGGGTAAAACTTTGTCTTAAGC
ATGGGCGTACTGTTCTTGGTTAAAAAAAAAAA
ATG and TGA are start and stop codons
respectively.

Full length fragments were cloned to pCAMBIA1302 vector using primers:

F: CCATGGTGAGAGGACTGCTCAA
R: ACTAGTTCATTTGTAAGCTTTCACTGCT

Primer F has NcoI (underlined) site whereas primer R has SpeI (underlined) site. Arabidopsis plants were used as initial transformation system.

### Transgenic Evaluation:

Wild type (hereinafter known as WT; i.e. control plant) along with two lines of transgenic Arabidopsis (NG3, NG15) were analyzed for gene expression and stress tolerance. Seeds obtained after T2 (second generation of transgenic) stage were germinated on Murashige and Skoog Agar (hereinafter known as MSA) plates and kept at 4° C., and 20° C. Gene expression was studied as follows:

### RNA Isolation, Quantification of RNA and Gel-Electrophoresis:

Ribonucleic acid (hereinafter, referred to “RNA”) from young leaf tissue of Arabidopsis Thaliana was isolated using iRIS Plant RNA Kit (Ghawana et al., US Patent no 0344NF2004/IN). Leaf tissue (100 mg) was ground in liquid nitrogen to fine powder using pre-chilled pestle and mortar. Solution I (2 ml) was added to the frozen powder and ground the mixture while still frozen (allow thawing with intermittent grinding) and thawed it completely. Solution II (800 μl) was added and ground for a while. Resulting homogenate was transferred to a 2 ml microcentrifuge tube and left undisturbed for 5 min at room temperature. Chloroform (200 μl) was added to each tube, vortexed briefly and left undisturbed for 10 min at room temperature. Centrifuged at 13,000 rpm for 10 min at 4° C. Transferred upper aqueous phase to a fresh tube (avoid contamination with interphase). Isopropanol (0.6 volume) was added, vortexed briefly and left undisturbed for 10 min at room temperature. Centrifuged at 13,000 rpm for 10 min at 4° C. Washed the RNA pellet with 70% ethanol (in DEPC-treated autoclaved water) by vortexing briefly followed by centrifugation at 13,000 rpm. Air dried the samples for 10-15 min and dissolved the pellet in 20-30 μl of DEPC-treated autoclaved water. RNA was quantified by measuring absorbance at 260 nm and the purity was monitored by calculating the ratio of absorbance measured at 260 and 280 nm. A value >1.8 at 260/280 nm was considered ideal for the purity of RNA used in the present investigation. The formula used to calculate RNA concentration and yield was as follows:

Concentration of RNA (μg/ml)=A₂₆₀ (absorbance at 260 nm)×40×dilution factor.

Total yield (μg)=concentration×volume of stock RNA sample.

To check the integrity of RNA, 5-6 μg of RNA in 4.5 μl of DEPC treated autoclaved water was diluted with 15.5 μl of Ml solution (2 μl of 5×MOPS buffer, 3.5 μl of formaldehyde, and 10 μl of formamide [5×MOPS buffer: 300 mM sodium acetate, 10 mM MOPS (3-{N-morpholino]propanesulfonic acid}, 0.5 mM ethylene diamine tetra-acetic acid (EDTA)] and incubated for 15 min at 65° C. RNA was loaded onto 1.0% formaldehyde agarose-gel after adding 2 μl of formaldehyde-gel loading buffer [50% glycerol, 1 mM EDTA (pH, 8.0), 0.25% bromophenol blue, 0.25% xylene cyanol FF], and electrophoresed at 72 volts in 1×MOPS buffer (60 mM sodium acetate, 2 mM MOPS, 0.1 mM EDTA), (Sambrook, J., Fritsch, E. F. and Maniatis, T. 1989. Molecular Cloning: A Laboratory Manual, Cold Spring Harbor Laboratory Press, Plainview, N.Y.).

Synthesis of Complementary DNA (Hereinafter Referred to “cDNA”):

cDNA was synthesized using total RNA preparations (2 μg) in the presence of 1 μg oligo(dT)₁₂₋₁₈ and 400 U of reverse transcriptase Superscript II (Invitrogen) after digesting with 2 U DNase I (amplification grade, Invitrogen, USA) following the manufacturer's instructions.

Amplification:

Expression primers which were used to amplify PCR products from cDNA template are as follows:

NF1 5′-CTTGGGCAGATACTGGTTAC-3′
NR1 5′-TTATCAGCAGTCTCCGCTAT-3′

PCR was performed using 1 μl cDNA template, 0.2 μM each of left primer and right primer, 0.2 μM of dNTPs, 1 Unit of Thermus aquaticus (hereinafter, referred to “Taq”) DNA polymerase (purchased from M/S. Qiagen, Germany), and 1×PCR buffer (20 mM Tris-HCl, pH 8.4, 50 mM KCl, 1.5 mM MgCl₂) in a final volume of 25 μl. Thermocycler program consisted of 27 cycles of initial denaturation at 94° C. for 5 min, followed by 94° C. for 30 sec, 55° C. for 40 sec and 72° C. for 1 min and then a final extension at 72° C. for 7 min. After the completion of PCR, 20 μl of PCR sample was run on 1.2% agarose gel in TAE buffer containing ethidium bromide (final concentration of 0.5 μg/ml).

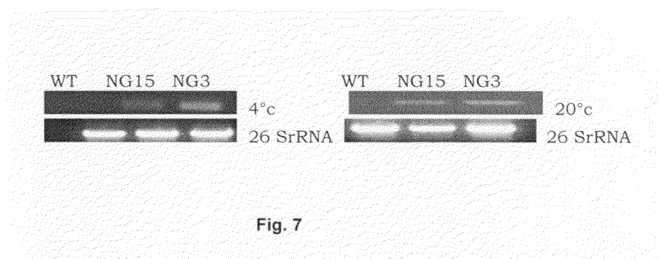

As is evident in FIG. 7, the gene did not express in wild type (WT) but expressed well in transgenic lines (NG3, NG15) of Arabidopsis as confirmed by RT-PCR.

FIG. 7 represents expression of gene (SEQ ID NO.1) in wild type (WT) and transgenic lines (NG3, NG15) of Arabidopsis.

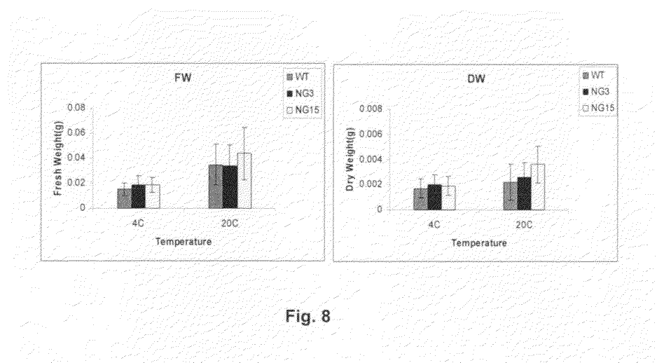

Further, transgenic lines always performed better in terms of accumulation of fresh weight or the dry weight (FIG. 8

For low temperature treatment, seeds were sown in petri plates containing MSA supplemented with 2% sucrose and 0.8% agar and kept at 4° C. for 2 days in dark and then transferred to 22° C. for 7 days. The 7-day-old seedlings were transferred to fresh MSA plates and allowed to grow for 3 days. After 21 days, fresh weight and dry weight were measured. For dry weight measurement plants were kept at 80° C. for 48 hrs.

FIG. 8 represents the effect of Low temperature on Fresh weight [FW] and Dry weight [α] of wild type (WT) and transgenic lines (NG3, NG15) of Arabidopsis.

To test root growth under stress conditions, seeds were grown in petri plates containing MSA supplemented with 2% sucrose and 0.8% agar and kept at 4° C. for 2 days in dark, later transferred to 22° C. for 7 days. The 7-day-old seedlings were transferred to fresh MSA plates and allowed to grow for 3 days. For 21 days plates were placed vertically to measure root length. With reference to 0 day, percent increase in root length at 4° C. for WT, NG15, NG3 was 35%, 40%, 33% respectively while at 20° C. it was 32%, 36%, 33% for the same.

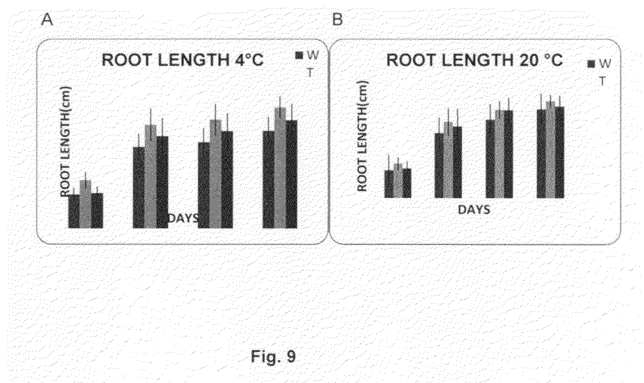

FIGS. 9A-9B represent the effect of cold stress on root length of transgenic plants (A) 4° C. (B) 20° C.

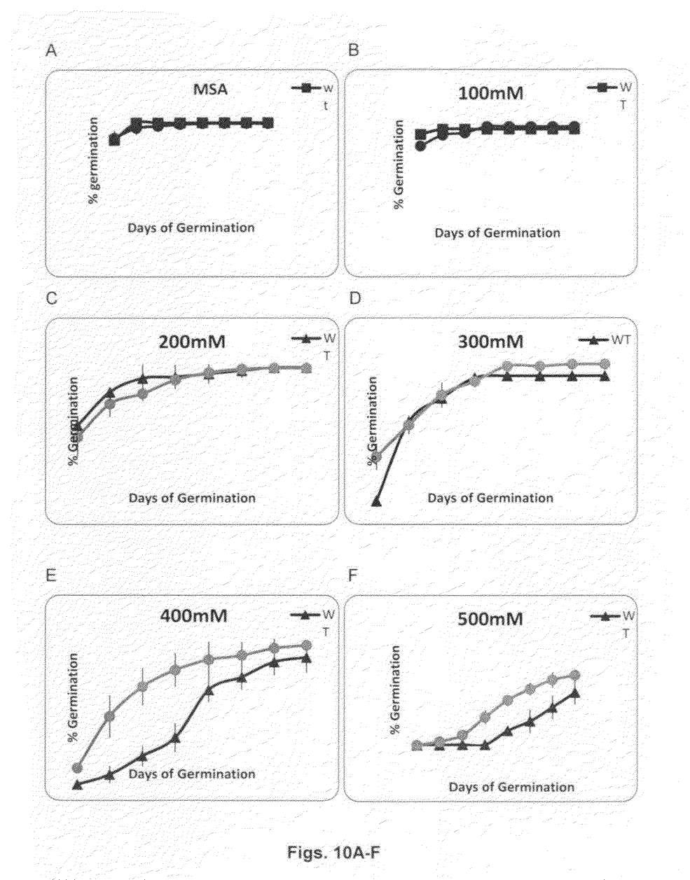

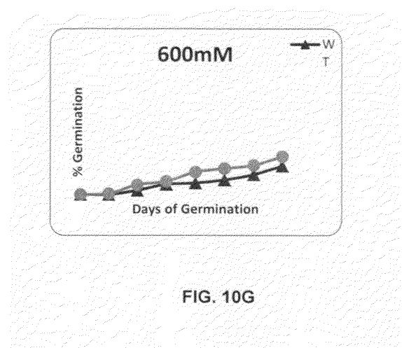

Under the condition of dehydration stress (FIGS. 10A-10F), transgenic lines outperformed particularly in terms of germination at higher concentrations of mannitol (i.e. higher levels of dehydration stress). As concentration of mannitol increased, decline in % germination was more in WT than the transgenic lines.

FIGS. 9A-9B represent the effect of dehydration stress on seed germination.

To determine the effect of salt on seed germination, MSA was supplemented with 100, 200 mM NaCl. No significant changes in germination rate were observed between WT and transgenic lines in 100 mM NaCl. When WT and transgenic lines (NG3,NG15) were germinated in presence of 200 mM NaCl, about 65% of WT seeds germinated by 8^{th} day, where as 80% of NG15, 50% of NG3 seeds had germinated by 8^{th} day.

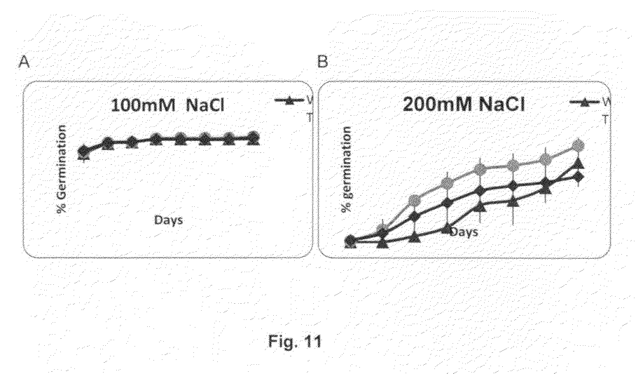

FIGS. 11A-11B represent the effect of salt stress on germination.

As described for Sequence ID 1, full-length cloning of this partial gene was also achieved except that the following primers were used for RACE:

Primers for 5′ RACE
RACE 1
5′CAAGTGCAAACCCCCAAGAAAGACAACA 3′
RACE 2
5′TGCAAACCCCCAAGAAAGACAACAACA 3′ (Nested)
Primers for3′ RACE
RACE 3
5′ TTGTTGTTGTCTTTCTTGGGGGTTTGC 3′
RACE 4
5′ TGTTGTCTTTCTTGGGGGTTTGCACTTG 3′ (Nested)
Full length Sequence (SEQ ID NO: 33):
ACGCGGGGAAGCATATCTACCAAAACTACTAAATTAGAAGCTCTAGTTCC
AAAAAAAATGGCTTCCATCAGTTTCTGGTCACTCATTTTACCACTTGTTG
TACTTCTAACATTTACAAGTGTTGAGGGGGCAAGGAATCTGCAACAATCA
ACATTGTCAAAAGTACATGAAGTTTCTAAAATTGATTTGCCTCCAGTTGC
AGGACTACCAAATCTTCCATTGCCACTTCCAAAACCAGAGTTGCCACCAC
TGCCAAATGTTCCAATCCCTGGTGTGCCTGTAAATGTAATTCCAACACTT
CCTACAATTCCTACAATACCTACAGTACCTGGTATTCCTGGTATTCCAAA
AGTGCCTACAATACCTGGCATTCCCAAGCCTGAGTTGCCTGCTGTGCCAA
AACCATAAGTACTAAAGTTCCTTATGTCTATCCCCAACTCTCTA
TTTTACTTTAGTAACATGTACTGTTAAATGTCTTGGTTATAATTTGTTGT
TTCTTTCTTGGGGGTTTGCACTTGTACTTTATGTATCACTTAATTACATG
TTATGTTCAGACGTTTTTAAAAAAAAAAA

The gene was cloned into pGEM-T easy vector (Promega) using the following primers:

TNG3 F 2 Nco1:
5′ TGCCATGGGCTTCCATCAGTTTCTGGTCACTC
TNG3 R 2 Spe1:
5′CGACTAGTTGGTTTTGGCACAGCAGGCAACTCAGG

Primer TNG3 F 2 NcoI has NcoI (underlined) site whereas primer TNG3 R 2 SpeI has SpeI (underlined) site to allow directional and in frame cloning into pCAMBIA 1302 which also has the same corresponding site.

Transgenic plants were generated as described above and the analysis was performed for the growth at low temperature. For the purpose seeds were sown on petri dishes following the standard procedure as mentioned above and the data on root growth was recorded since the performance of root determined the plant performance.

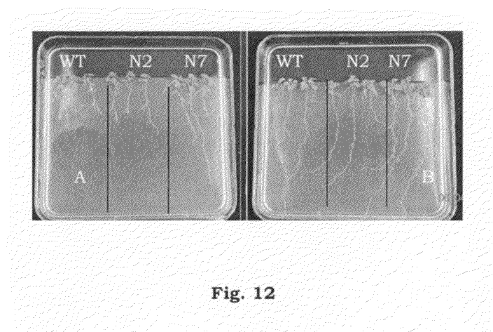

Data showed the improved performance of root growth of transgenics (N2, N7) at lower temperature (15° C.) as compared to the WT. Though at normal growth temperature, both WT and the transgenics behaved similarly (FIGS. 12-15).

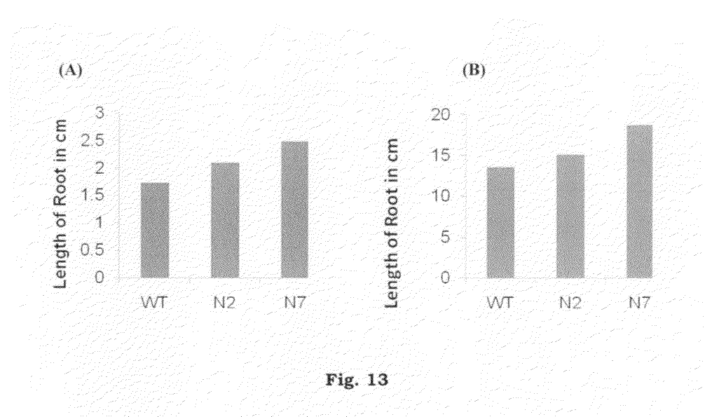

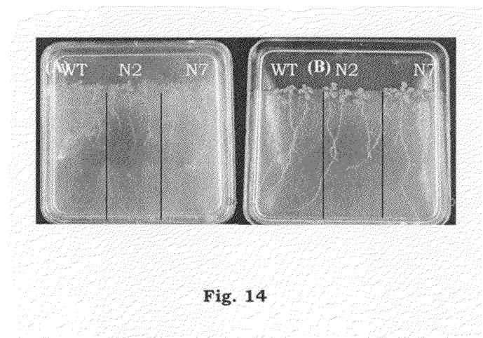

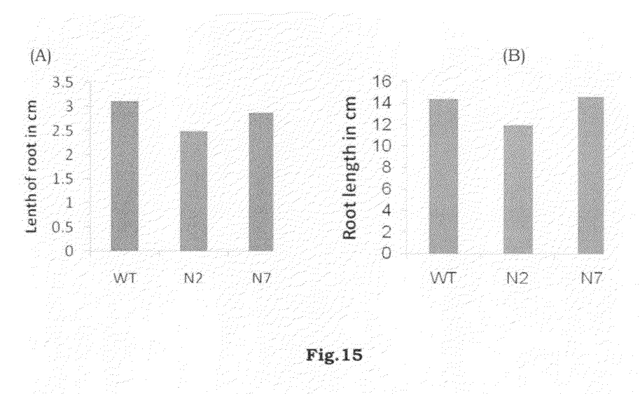

FIGS. 12A-12B show root growth at 15° C. after (A) 1 Week, (B) 2 Weeks
FIGS. 13A-13B show root length at 15° C. after (A) 1 Week (B) 2 Weeks
FIGS. 14A-14B show root growth at 22° C. after (A) 1 Week, (B) 2 Weeks
FIGS. 15A-15B show root length at 22° C. after (A) 1 Week (B) 2 Weeks

### How the EST was Used to Clone the Full Length Gene:

Sequence ID 32 was an EST that represented the extreme 3′ end of the gene. This region is not translated into protein. Part of the gene that participated in translational event lies between start (ATG) and stop codon (TAA). There we used a part of the Sequence ID 32 sequence to design primers for RACE to clone the full length genes.

The sequence of the primer was

5′ CAAGTGCAAACCCCCAAGAAAGACAACA 3′

This sequence is mentioned in our response.

This sequences is reverse complementary to the sequence present in EST sequence ID 32 as follows:

Sequence 32 [Sequence 3 (273 bp)]
5′ AAGCGAGACTGCAGTGAGCAGAGACGTAGCTACAGTGCAGCAGCACT
GACGAGTACACTCATCAACATCGACTGATCTGATCAAGGCTCATCTGCAT
CAGCTGAGTGCGTGCTGTGACTGACAAGTACAAGTCTATGTCTATCCCTA
CTCTCTATTTACTTTAGTAACATGTACTGTTAAATGTCTTGGTATAATTT
GTTGTTGTCTTTCTTGGGGGTTTGCACTTGTACTTTATGTATCACTTAAT
TA CATGTTATGT TCAGACGTTTTT 3′
Reverse compliment of Sequence 32. (The portion
where the primer appears is shown in red)
AAAAACGTCTGAACATAACATGTAATTAAGTGATACATAAAGTACAAGTG
CAAACCCCCAAGAAAGACAACAACAAATTATACCAAGACATTTAACAGTA
CATGTTACTAAAGTAAATAGAGAGTAGGGATAGACATAGACTTGTACTTG
TCAGTCACAGCACGCACTCAGCTGATGCAGATGAGCCTTGATCAGATCAG
TCGATGTTGATGAGTGTACTCGTCAGTGCTGCTGCACTGTAGCTACGTCT
CTGCTCACTGCAGTCTCGCTT
Using the RACE primers as described above, we got
the complete gene sequence (SEQ ID NO: 33) with
ATG and TAA as follows.
ACGCGGGGAAGCATATCTACCAAAACTACTAAATTAGAAGCTCTAGTTCC
AAAAAAAATGGCTTCCATCAGTTTCTGGTCACTCATTTTACCACTTGTTG
TACTTCTAACATTTACAAGTGTTGAGGGGGCAAGGAATCTGCAACAATCA
ACATTGTCAAAAGTACATGAAGTTTCTAAAATTGATTTGCCTCCAGTTGC
AGGACTACCAAATCTTCCATTGCCACTTCCAAAACCAGAGTTGCCACCAC
TGCCAAATGTTCCAATCCCTGGTGTGCCTGTAAATGTAATTCCAACACTT
CCTACAATTCCTACAATACCTACAGTACCTGGTATTCCTGGTATTCCAAA
AGTGCCTACAATACCTGGCATTCCCAAGCCTGAGTTGCCTGCTGTGCCAA
AACCATAAGTACTAAAGTTCCTTATGTCTATCCCCAACTCTCTA
TTTTACTTTAGTAACATGTACTGTTAAATGTCTTGGTTATAATTTGTTGT
TTCTTTCTTGGGGGTTTGCACTTGTACTTTATGTATCACTTAATTACATG
TTATGTTCAGACGTTTTTAAAAAAAAAAA

The portion of the gene spanning between ATG and TAA was used for transgenic experiments

While this invention has been described as having preferred sequences, ranges, steps, materials, structures, components, features, and/or designs, it is understood that it is capable of further modifications, uses, and/or adaptations of the invention following in general the principle of the invention, and including such departures from the present disclosure as those come within the known or customary practice in the art to which the invention pertains, and as may be applied to the central features hereinbeforesetforth, and fall within the scope of the invention and of the limits of the appended claims.

The main advantages of the present invention:
(a) The main advantage of the present invention is that the genes responsible for freeze tolerance have been identified from field conditions.
(b) Another main advantage of the present invention is that the region of SEQ ID NOs: 30-33 responsible for variation in the sequence are identified by differential gene expression technique.
(c) Yet another advantage of the present invention is development of a method of introducing freeze tolerance in life forms by transforming them with said DNA sequences.
(d) Still another advantage is Novel genes expressed in the apical buds of Caragana plants growing under snow in natural environment have been cloned.
(e) Still another advantage is a method to clone the genes related to freezing temperature
(f) Still another advantage is a spectra of 3′ ends of the expressed and repressed genes in CO and SN apical buds of Caragana growing under field conditions for identification of differentially expressed genes has been presented.
(g) Still another advantage is confirmation of the identified 3′ ends of the differentially expressed gene(s) for establishing differential expression in the apical buds of Caragana experiencing freezing temperatures bush growing under field conditions has been carried out.
(h) Still another advantage is sequencing of the cloned 3′ ends of the differentially expressed gene(s) showed uniqueness in terms of novel sequences not deposited in the data bank so far.

1. An isolated DNA as set forth in SEQ ID NO: 33.


SEQUENCE LISTING (32 sequences)

SEQ ID NO: 1 - DNA, 18 bp, Artificial Sequence
  T11A anchored primers for differential display
aagcttttttttttttta

SEQ ID NO: 2 - DNA, 18 bp, Artificial Sequence
  T11C (anchored) primer for differential display
aagctttttttttttttc

SEQ ID NO: 3 - DNA, 18 bp, Artificial Sequence
  T11G (anchored) primer for differential display
aagctttttttttttttg

SEQ ID NO: 4 - DNA, 13 bp, Artificial Sequence
  AP1 arbitrary primer for differential display
aagcttgattgcc

SEQ ID NO: 5 - DNA, 13 bp, Artificial Sequence
  AP2 arbitrary primer for diferential display
aagcttcgactgt

SEQ ID NO: 6 - DNA, 13 bp, Artificial Sequence
  AP3 arbitrary primer for differential display
aagctttggtcag

SEQ ID NO: 7 - DNA, 13 bp, Artificial Sequence
  AP4 arbitrary primer for differential display
aagcttctacacg

SEQ ID NO: 8 - DNA, 13 bp, Artificial Sequence
  AP5 arbitrary primer for differential display
aagcttagtaggc

SEQ ID NO: 9 - DNA, 13 bp, Artificial Sequence
  AP6 arbitrary primer for differential display
aagcttgcaccat

SEQ ID NO: 10 - DNA, 13 bp, Artificial Sequence
  AP7 arbitrary primer for differential display
aagcttaacgagg

SEQ ID NO: 11 - DNA, 13 bp, Artificial Sequence
  AP8 arbitrary primer for differential display
aagcttttaccgc

SEQ ID NO: 12 - DNA, 13 bp, Artificial Sequence
  AP33 arbitrary primer for differential display
aagcttgctgctc

SEQ ID NO: 13 - DNA, 13 bp, Artificial Sequence
  AP34 arbitrary primer for differential display
aagcttcagcagc

SEQ ID NO: 14 - DNA, 13 bp, Artificial Sequence
  AP35 arbitrary primer for differential display
aagcttcagggca

SEQ ID NO: 15 - DNA, 13 bp, Artificial Sequence
  AP36 arbitrary primer for differential display
aagcttcgacgct

SEQ ID NO: 16 - DNA, 13 bp, Artificial Sequence
  AP37 arbitrary primer for differential display
aagcttgggccta

SEQ ID NO: 17 - DNA, 13 bp, Artificial Sequence
  AP38 arbitrary primer for differntial display
aagcttccagtgc

SEQ ID NO: 18 - DNA, 13 bp, Artificial Sequence
  AP39 arbitrary primer for differential display
aagctttcccagc

SEQ ID NO: 19 - DNA, 13 bp, Artificial Sequence
  AP40 arbitrary primer for differential display
aagcttgtcagcc

SEQ ID NO: 20 - DNA, 13 bp, Artificial Sequence
  AP65 arbitrary primer for differential display
aagcttcaagacc

SEQ ID NO: 21 - DNA, 13 bp, Artificial Sequence
  AP66 arbitrary primer for differential display
aagcttgccttta

SEQ ID NO: 22 - DNA, 13 bp, Artificial Sequence
  AP67 arbitrary primer for differential display
aagctttatttat

SEQ ID NO: 23 - DNA, 13 bp, Artificial Sequence
  AP68 arbitrary primer for differential display
aagcttctttggt

SEQ ID NO: 24 - DNA, 13 bp, Artificial Sequence
  AP69 arbitrary primer for differential display
aagcttaataacg

SEQ ID NO: 25 - DNA, 13 bp, Artificial Sequence
  AP70 arbitrary primer for differential display
aagctttcatatg

SEQ ID NO: 26 - DNA, 13 bp, Artificial Sequence
  AP72 arbitrary primer for differential display
aagcttgtagtaa

SEQ ID NO: 27 - DNA, 13 bp, Artificial Sequence
  AP72 arbitrary primer for differential display
aagctttcaaaga

SEQ ID NO: 28 - DNA, 17 bp, Artificial Sequence
  Lgh primer specific to the vector sequence flanking the cloning site
cgacaacaccgataatc

SEQ ID NO: 29 - DNA, 17 bp, Artificial Sequence
  Rgh primer specific to the vector sequence flanking the cloning site
gacgcgaacgaagcaac

SEQ ID NO: 30 - DNA, 211 bp, Caragana jubata
ggtacatatatcagagagcaatgagtgctagatcgtcatatgatacgcagtgctatcaaactgttccgaagtagtgcatttttcaaatttagttcaatggttgattcacgtggttgctgcattgctctctcttgcaactcaagatactgtccctttctgcgttagggctaaaactttgtcttaagcattggatgcgctactgttcttggtt

SEQ ID NO: 31 - DNA, 181 bp, Caragana jubata
cgagagcactacgtgcatatcggactgagtagcagtgtgtaggctagcatgtatgctgagtcgtaatgaactggaatgtctagtgttagagcaaatgcatgatgtatcctgtgatacatatattcgagtctagtctatcttagcaaattcttaatattaatttcttggaatccttactttt

SEQ ID NO: 32 - DNA, 271 bp, Caragana jubata
aagcgagactgcagtgagcagagacgtagctacagtgcagcagcactgacgagtacactcatcaacatcgactgatctgatcaaggctcatctgcatcagctgagtgcgtgctgtgactgacaagtacaagtctatgtctatccctactctctatttactttagtaacatgtactgttaaatgtcttggtataatttgttgttgtctttcttgggggtttgcacttgtactttatgtatcacttaattacatgttatgttcagacgttttt